(19) United States
(12) Patent Application Publication
(10) Pub. No.: US 2024/0384324 A1
(43) Pub. Date: Nov. 21, 2024

(54) METHODS OF DETERMINING DNA BARCODES FOR EFFICIENT SPECIES CATEGORIZATION USING NANOPORE TRANSLOCATION

(71) Applicant: University of Central Florida Research Foundation, Inc., Orlando, FL (US)

(72) Inventors: Aniket Bhattacharya, Orlando, FL (US); Swarnadeep Seth, Orlando, FL (US)

(21) Appl. No.: 18/787,273

(22) Filed: Jul. 29, 2024

Related U.S. Application Data
(63) Continuation of application No. 17/649,577, filed on Feb. 1, 2022, now Pat. No. 12,049,663.
(60) Provisional application No. 63/199,898, filed on Feb. 1, 2021.

(51) Int. Cl. C12Q 1/68; G06F 17/18

(52) U.S. Cl. CPC C12Q 1/68; G06F 17/18

(57) ABSTRACT

Described herein relates to methods of accurately determining DNA barcodes using a cylindrical nanopore system. The system and method may include the steps of leveraging the average velocity of a double-stranded DNA segment passing through a single cylindrical nanopore that may be measured through repeated scanning to accurately determine protein tag locations on the double-stranded DNA segment. As such, the system and methods may provide for the accurate calculation of a barcode for the double-stranded DNA segment based on protein tag locations without underestimation or overestimate issues. Additionally, the underlying concept and/or the system and/or the methods may be equally applicable to other multi-nanopore systems which use the dwell time and/or time of flight velocities to measure the barcodes.

### CROSS-REFERENCE TO RELATED APPLICATIONS

This nonprovisional patent application is a continuation of and claims the benefit of U.S. Nonprovisional patent application Ser. No. 17/649,577 entitled “METHODS OF DETERMINING DNA BARCODES FOR EFFICIENT SPECIES CATEGORIZATION USING NANOPORE TRANSLOCATION” filed Feb. 1, 2022 by the same inventors, which claims the benefit of and priority to U.S. Provisional Patent Application No. 63/199,898 entitled “METHODS OF DETERMINING DNA BARCODES FOR EFFICIENT SPECIES CATEGORIZATION USING NANOPORE TRANSLOCATION” filed Feb. 1, 2021 by the same inventors, all of which are incorporated herein by reference, in their entireties, for all purposes.

### FEDERALLY SPONSORED RESEARCH OR DEVELOPMENT

This invention was made with Government support under 1R21HG011236-01 as awarded by the National Human Genome Research Institute at the National Institutes of Health. The government has certain rights in the invention.

### BACKGROUND OF THE INVENTION

### 1. Field of the Invention

This invention relates, generally, to methods of species categorization. More specifically, it relates to methods of determining DNA barcodes for efficient species categorization without relying on traditional chemical-based DNA sequencing of lengthy sections of nucleotides.

### 2. Brief Description of the Prior Art

The methods of DNA sequencing and species categorization provide essential insight into basic biological research for studied species, and help describe relationships between different species. The knowledge gained through DNA sequencing techniques is useful across a broad scientific spectrum, such as by conserving biodiversity [1],estimating phyletic diversity, identifying disease vectors [2], authenticating herbal products [3], unambiguously labeling food products [4, 5], and protecting endangered species [1]. Rather than sequencing an entire DNA strand, researchers determined that DNA barcodes could be determined based on a targeted gene, and that these barcodes yield accurate species identifications. A DNA barcode consists of a short strand of DNA sequence taken from a targeted gene like COI or cox I gene (Cytochrome C Oxidase 1) [6] present in the mitochondrial gene in animals. As such, during the early 21^{st} century, DNA sequencing techniques dramatically improved as quicker categorizations were possible based on these DNA barcodes.

To determine the DNA barcode, traditional sequencing methods based on chemical analyses are widely used in the biological community. Recently, nanopore-based sequencing methods [7] have been explored in a dual nanopore system for a cost effective, high throughput, chemical-free, and real time barcode generation. Dual nanopore systems determine DNA barcodes by scanning a captured dsDNA (double stranded DNA) multiple times as the strand passes through both pores of the dual nanopore system, applying a net periodic bias across the two pores. However, such a system relies on the accurate calculation of dwell time or time of flight (hereinafter “TOF”') of the barcodes (e.g., tags) using the current blockage information from individual nanopores. As the tags are heavier and bulkier in nature, they produce significant current blockage (e.g., increased dwell time) compared to the normal nucleotide monomers. The disparate velocity of tags and monomers within a segment leads to an over/underestimation of the distance between sequential tags if only dwell or TOF velocity information is used.

Accordingly, what is needed is an improved method of DNA barcoding to efficiently categorize species without suffering from over/underestimation problems related to the distance between measured tags within the DNA sequence. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned

### SUMMARY OF THE INVENTION

The long-standing but heretofore unfulfilled need for a method of categorizing a species associated with a segment of double-stranded DNA is now met by a new, useful, and nonobvious invention.

The novel method includes a step of passing a segment of double-stranded DNA through a singular cylindrical nanopore formed within a test chamber. The segment of double-stranded DNA may include a plurality of monomers, a first protein tag, and/or a subsequent protein tag. Each of the plurality of monomers and each protein tag may have an equal size, shape, and/or volume. In an embodiment, the test chamber may also include at least two opposing longitudinal walls joined together by at least two opposing lateral walls, such that the singular cylindrical nanopore may be formed between the two opposing longitudinal walls, such that a central axis of the singular cylindrical nanopore may be parallel to each of the at least two opposing lateral walls. In an embodiment, the singular cylindrical nanopore may comprise an associated diameter of 2σ, where σ is a diameter of each of the plurality of monomers, the first protein tag, and/or the subsequent protein tag.

In some embodiments, the method may also include a step of calculating an average scanning velocity of the segment of double-stranded DNA by dividing a length of the segment of double-stranded DNA by an average scanning time for the double-stranded DNA taken for multiple scans. In these other embodiments, the method comprises a step of retaining at least a portion of the segment of double-stranded DNA within the singular cylindrical nanopore throughout each of the multiple scans.

In some embodiments, the method may include a step of passing the segment of double-stranded DNA through the singular cylindrical nanopore in an opposing direction and/or repeating the steps of calculating the average scanning velocity, calculating the estimated distance between the first protein tag and/or the subsequent protein tag, calculating the estimated number of monomers of the plurality of monomers, calculating the weighted velocity of the segment of double-stranded DNA, and/or calculating the distance between the first protein tag and the subsequent protein tag. In addition, a bias voltage may be applied to the test chamber in a reverse direction prior to passing the segment of double-stranded DNA through the singular cylindrical nanopore in the opposing direction.

An estimated distance between a first protein tag and a subsequent protein tag of the segment of double-stranded DNA may be calculated by measuring, for the first protein tag and/or the subsequent protein tag, a dwell time and/or a dwell velocity based on an entry time into the singular cylindrical nanopore and/or an exit time from the singular cylindrical nanopore. In some embodiments, using the estimated distance between the first protein tag and the subsequent protein tag, an estimated number of monomers of the plurality of monomers that are disposed between the first protein tag and the subsequent protein tag may also be calculated.

In some embodiments, a weighted velocity of the segment of double-stranded DNA may be calculated using the dwell velocity for each of the first protein tag and/or the subsequent protein tag, the average scanning velocity of the segment of double-stranded DNA, and/or the estimated number of monomers. In these other embodiments, the weighted velocity of the segment of double-stranded DNA may be calculated using

$${v}_{weight}^{U \to D} = \frac{1}{{N}_{mn}} [ {v}_{dwell}^{U \to D} ( m ) + {v}_{dwell}^{U \to D} ( n ) + ( {N}_{mn} - 2 ) {\overline{v}}_{scan} ] ,$$

where v_{weight}^{U→D} may represent the weighted velocity in a downward direction through the singular cylindrical nanopore, Nₘₙ may represent the estimated number of monomers of the plurality of monomers, v_{dwell}^{U→D}(m) may represent the dwell velocity of the first protein tag in the downward direction through the singular cylindrical nanopore, v_{dwell}^{U→D}(n) may represent the dwell velocity of the subsequent protein tag in the downward direction through the singular cylindrical nanopore, and/or v̅_{scan} may represent the calculated average scanning velocity of the segment of double-stranded DNA.

In some embodiments, the method may further comprise a step of passing the segment of double-stranded DNA through the singular cylindrical nanopore in an opposing direction. As such, the weighted velocity of the segment of double-stranded DNA in an upward direction through the singular cylindrical nanopore may be calculated using

$${v}_{weight}^{D \to U} = \frac{1}{{N}_{mn}} [ {v}_{dwell}^{D \to U} ( m ) + {v}_{dwell}^{D \to U} ( n ) + ( {N}_{mn} - 2 ) {\overline{v}}_{scan} ] .$$

Moreover, in some embodiments, the method may comprise a step of calculating the distance between the first protein tag and the subsequent protein tag by multiplying the weighted velocity of the segment of double-stranded DNA by a time delay between the entry time of the first protein tag and the entry time of the subsequent protein tag. In these other embodiments, the steps of calculating a distance between sequential protein tags may be repeated for a plurality of protein tags within the segment of double-stranded DNA.

In some embodiments, the novel method may also include a step of applying a first voltage to a first side of a test chamber that defines a singular nanopore therethrough. In these other embodiments, based on the applied first voltage, the method may further comprise a step of passing the segment of double-stranded DNA through the first side of the singular nanopore defined by the test chamber. In this manner, the method may further comprise a step of applying a second voltage to a second side of the test chamber, with the second side of the test chamber being opposite the first side of the test chamber, such that a bias voltage applied to the test chamber may reverse. As such, based on the applied second voltage, the method may comprise a step of passing the segment of double-stranded DNA through the second side of the singular nanopore in a direction toward the first side of the test chamber.

In some embodiments, the novel method may further comprise the step of calculating the distance between the first protein tag and the subsequent protein tag for each of a plurality of protein tags on a segment of dsDNA. In some embodiments, the method may also include a step of generating a barcode for the segment of double-stranded DNA by arranging the plurality of protein tags of the segment of double-stranded DNA in sequential order.

An object of the invention is to provide efficient and accurate methods of calculating distances between sequential protein tags of a double-stranded DNA, thereby providing for efficient categorization of species based on the calculated DNA barcode.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not restrictive.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A depicts a schematic diagram depicting a dsDNA strand being scanned through a cylindrical nanopore device translocating in the direction of the bias net force ±|Δ\vec{f}_{UD}|=±|\vec{f}_{U}−\vec{f}_{D}|, according to an embodiment of the present disclosure.

FIG. 1B depicts an example of the positions of protein tags (T₁ through T₈) along the contour length of a model dsDNA, according to an embodiment of the present disclosure.

FIG. 1C depicts an electrical schematic diagram showing an applied voltage on a first side of a nanopore (V_{T}) and an applied voltage on a second side of the nanopore (V_{B}), such that a bias net force can be reversed, according to an embodiment of the present disclosure.

FIG. 2 depicts an embodiment of a dsDNA translocating through a cylindrical pore, showing a bias net force of Δ\vec{f}_{DU}=\vec{f}_{D}−\vec{f}_{U}>0 on the left side, and showing a bias net force of Δ\vec{f}_{UD}=\vec{f}_{U}−\vec{f}_{D}>0 on the right side, according to an embodiment of the present disclosure.

FIG. 3 depicts a graphical representation of measuring the dwell velocity and tag time delay between two tags (T₇ and T₈) using a cylindrical nanopore, according to an embodiment of the present disclosure.

FIG. 4 depicts the dwell velocity of the monomers in a downward translocation direction U→D (downward facing triangles), in an upward translocation direction D→U (upward facing triangles), and the corresponding averaged velocities (circles), according to an embodiment of the present disclosure.

FIG. 5 depicts an example of tension propagation within a DNA strand, specifically showing the quicker passage of monomers through a pore, according to an embodiment of the present disclosure.

FIG. 6A depicts experimental results of a calculated DNA barcode, according to an embodiment of the present disclosure.

FIG. 6B depicts experimental results of a DNA barcode generated using measured dwell velocities of tags with a known end-to-end tag distance in a single nanopore device, according to an embodiment of the present disclosure.

FIG. 6C depicts experimental results of a DNA barcode generated using measured dwell velocities of tags using an average scan time of an entire strand in a single nanopore device, according to an embodiment of the present disclosure.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention.

As such, elements/components shown in diagrams are illustrative of exemplary embodiments of the disclosure and are meant to avoid obscuring the disclosure. Any headings, used herein, are for organizational purposes only and shall not be used to limit the scope of the description or the claims.

Furthermore, the use of certain terms in various places in the specification, described herein, are for illustration and should not be construed as limiting. For example, any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Therefore, a reference to first and/or second elements does not mean that only two elements may be employed there or that the first element must precede the second element in some manner. Also, unless stated otherwise a set of elements may comprise one or more elements

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

Referring in general to the following description and accompanying drawings, various embodiments of the present disclosure are illustrated to show its structure and method of operation. Common elements of the illustrated embodiments may be designated with similar reference numerals.

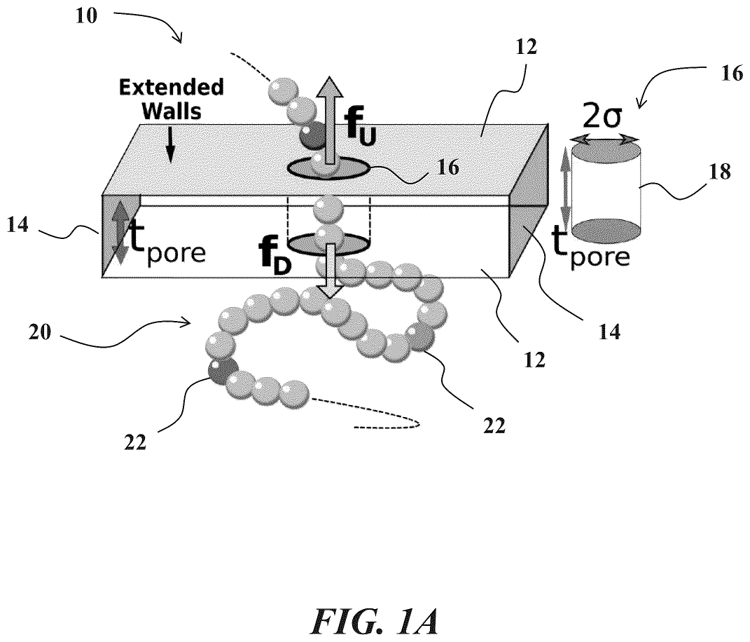

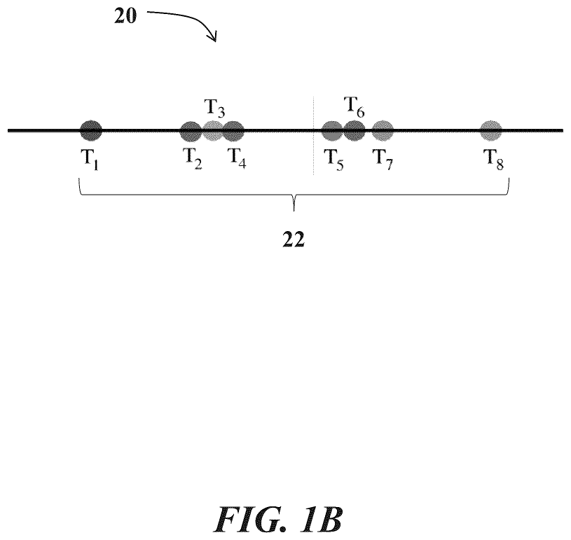

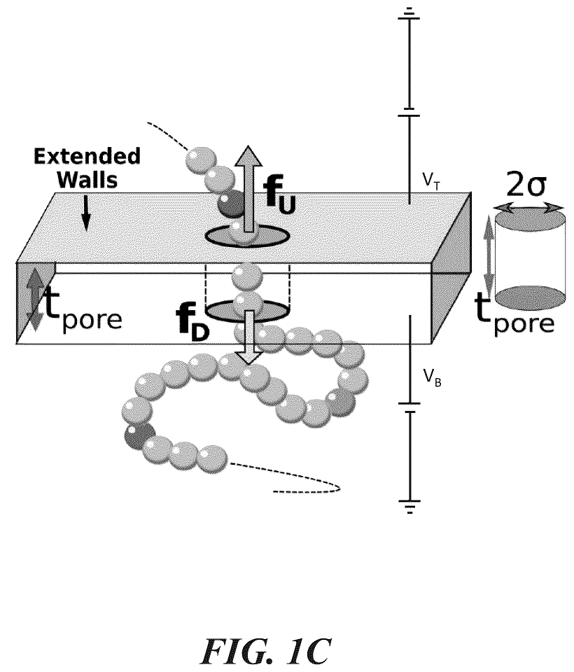

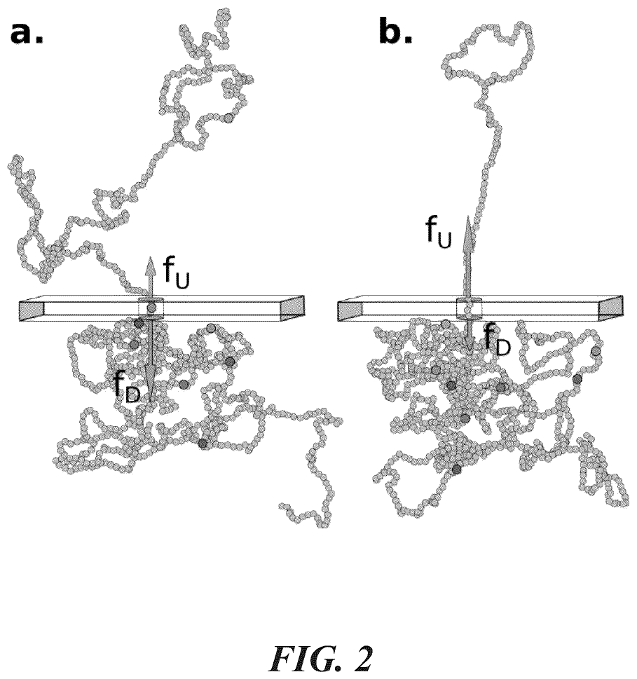

Accordingly, the relevant descriptions of such features apply equally to the features and related components among all the drawings. For example, any suitable combination of the features, and variations of the same, described with components illustrated in FIG. 1, can be employed with the components of FIG. 2, and vice versa. This pattern of disclosure applies equally to further embodiments depicted in subsequent figures and described hereinafter. It should be understood that the figures presented are not meant to be illustrative of actual views of any particular portion of the actual structure or method but are merely idealized representations employed to more clearly and fully depict the present invention defined by the claims below.

### Definitions

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

In the following description, for the purposes of explanation, numerous specific details are set forth in order to provide a thorough understanding of embodiments of the present technology. It will be apparent, however, to one skilled in the art that embodiments of the present technology may be practiced without some of these specific details. The techniques introduced here can be embodied as special-purpose hardware (e.g. circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compacts disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

As used herein, the term “communicatively coupled” refers to any coupling mechanism known in the art, such that at least one electrical signal may be transmitted between one device and one alternative device. Communicatively coupled may refer to Wi-Fi, Bluetooth, wired connections, wireless connection, and/or magnets. For ease of reference, the exemplary embodiment described herein refers to Wi-Fi and/or Bluetooth, but this description should not be interpreted as exclusionary of other electrical coupling mechanisms.

As used herein, the terms “about,” “approximately,” or “roughly” refer to being within an acceptable error range (i.e., tolerance) for the particular value as determined by one of ordinary skill in the art, which will depend in part on how the value is measured or determined (e.g., the limitations of a measurement system) (e.g., the degree of precision required for a particular purpose, such as determining DNA barcodes for efficient species categorization without relying on traditional chemical-based DNA sequencing of lengthy sections of nucleotides). As used herein, “about,” “approximately,” or “roughly” refer to within ±25% of the numerical.

All numerical designations, including ranges, are approximations which are varied up or down by increments of 1.0, 0.1, 0.01 or 0.001 as appropriate. It is to be understood, even if it is not always explicitly stated, that all numerical designations are preceded by the term “about”. It is also to be understood, even if it is not always explicitly stated, that the compounds and structures described herein are merely exemplary and that equivalents of such are known in the art and can be substituted for the compounds and structures explicitly stated herein.

Wherever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “no more than,” “less than,” or “less than or equal to” precedes the first numerical value in a series of two or more numerical values, the term “no more than,” “less than” or “less than or equal to” applies to each of the numerical values in that series of numerical values. For example, less than or equal to 1, 2, or 3 is equivalent to less than or equal to 1, less than or equal to 2, or less than or equal to 3.

### System of and Methods for Determining DNA Barcode(s)

The present invention pertains to methods of accurately determining DNA barcodes using a cylindrical nanopore as opposed to a dual nanopore architecture. The methods of the present invention explain the underestimation of DNA tags caused by the fast-moving nucleotides in between the barcodes of a strand using tension propagation theory [8]. Instead, the methods described herein, schematic and graphical diagrams of which are shown in FIGS. 1A-3 and FIG. 5, leverage the average velocity of a dsDNA segment passing through a single cylindrical nanopore measured through repeated scanning to accurately determine tag locations to barcode the dsDNA segment without the underestimation issues of the prior art. These methods are described in greater detail herein below.

As shown in particular in FIG. 1A, in an embodiment, dsDNA test chamber 10 may include a body that is defined by dual opposing longitudinal walls 12 and/or dual opposing lateral walls 14, with each longitudinal wall 12 secured to each lateral wall 14, such that opposing longitudinal walls 12 may be spaced apart from each other, and/or such that opposing lateral walls 14 may be spaced apart from each other. The body of dsDNA test chamber 10 may define nanopore 16, therethrough, with nanopore 16 spanning between opposing longitudinal walls 12, such that a central axis of nanopore 16 may be approximately aligned with opposing lateral walls 14. As such, in this embodiment, the body of dsDNA test chamber 10 may also include one or more interior lateral walls 18 that define nanopore 16 therebetween. In this embodiment, dsDNA test chamber 10 may further include a single interior lateral wall 18, such that the defined nanopore 16 may be cylindrical in shape; however, it should be appreciated that in alternative embodiments, nanopore 16 may be defined as geometrical shapes including polygonal wall orientations, such as triangular, rectangular, pentagonal, hexagonal, and/or the like.

In addition, still referring to FIG. 1A, in an embodiment, a diameter of the defined nanopore 16 (e.g., the distance between the one or more interior lateral walls 18) may measure approximately 2σ, where σ may comprise the diameter of each monomer or tag present on a segment of dsDNA passing through nanopore 16. In this manner, during translocation through nanopore 16, a time may be measured for the passage of each tag from a first end of nanopore 16 (e.g., defined by a first wall of opposing longitudinal walls 12) and/or a second end of nanopore 16 (e.g., defined by a second wall of opposing longitudinal walls 12). For example, in some embodiments, (e.g., in the embodiment shown in FIG. 1B), a plurality of tags 22 (e.g., T₁ through T₈) of dsDNA 20 may be spaced apart from one another, such that a translocation time of each tag 22 may be determined during passage through nanopore 16. The time of the translocation for each tag 22 may be defined as the dwell time W(m) and/or may be used to determine a dwell velocity of each tag 22, as will be described in greater detail below.

Similar to a double nanopore setup, in an embodiment, the single cylindrical nanopore 16 may comprise a periodical variation in the differential bias applied at nanopore 16 to scan the co-captured DNA multiple times. The force bias direction may be altered when either of the end tags is detected at the nanopore preventing the DNA chain from escaping the nanopore for a long time. As such, in some embodiments, (e.g., the embodiment shown in FIG. 2), as a segment of dsDNA passes through nanopore 16 in a direction with tag 22 T₁ passing through nanopore 16 first and tag 22 T₈ passing through nanopore 16 last, a downward bias net force of Δ\vec{f}_{DU}=\vec{f}_{D}−\vec{f}_{U}>0 may be configured to act on the dsDNA until tag 22 T₈ traverses through nanopore 16. After tag 22 T₈ traverses through nanopore 16 and each of the plurality of tags 22 pass through nanopore 16, a bias voltage may then be applied to dsDNA test chamber 10 reverses such that an upward bias net force of Δ\vec{f}_{UD}=\vec{f}_{U}−\vec{f}_{D}>0 may act on the dsDNA until tag 22 T₁ completes a translocation through nanopore 16, at which time the bias voltage may be applied to dsDNA test chamber 10 reverses again (e.g., shown in detail in FIG. 1C). As such, dsDNA test chamber 10 may provide for the repeated scanning of each of the plurality of tags 22 to obtain an accuracy averaged dwell time and/or dwell velocity for each tag 22.

As shown in FIG. 1C, in an embodiment, dsDNA test chamber 10 may also comprise a first voltage source (e.g., labeled as V_{T}) opposite a second voltage source (e.g., labeled as V_{B}). In this manner, the first voltage source V_{T} may be in electrical communication with a first wall of opposing longitudinal walls 12; similarly, the second voltage source V_{B} may be in electrical communication with a second wall of opposing longitudinal walls 12. As such, the opposing voltage sources V_{T} and/or V_{B} may be disposed on opposite sides of nanopore 16. Accordingly, in this embodiment, as the plurality of tags 22 translocate through nanopore 16 in a direction with tag 22 T₁ passing through nanopore 16 first and tag 22 T₈ passing through nanopore 16 last, with the downward bias net force of Δ\vec{f}_{DU}=\vec{f}_{D}−\vec{f}_{U}>0 acting on the dsDNA, the applied second voltage source V_{B} may be greater than or equal to the applied first voltage source V_{T}. After tag 22 T₈ passed through nanopore 16, the bias voltage may reverse, such that the applied first voltage source V_{T} may be greater than or equal to the applied second voltage source V_{B}.

Moreover, as shown in FIG. 1C, in an embodiment, a feature may be added to opposing ends of a strand of dsDNA 20, such that one feature is disposed proximate to tag 22 T₁, with tag 22 T₁ being disposed between the feature and tag 22 T₂; similarly, the other feature may be disposed proximate to tag 22 T₈, with tag 22 T₈ being disposed between the feature and tag 22 T₇. As such, upon the passage of one of the features through an end of nanopore 16, the bias voltage may reverse, thereby allowing the reverse scanning of the strand of dsDNA 20 via a flossing technique. It should be appreciated that other methods of reversing bias voltages may be used in combination with dsDNA test chamber 10, such as utilizing field programmable gate arrays to input a control logic to automatically reverse the bias voltage and recapture scanned tags 22 by progressively increasing the number of tags 22 scanned during flossing.

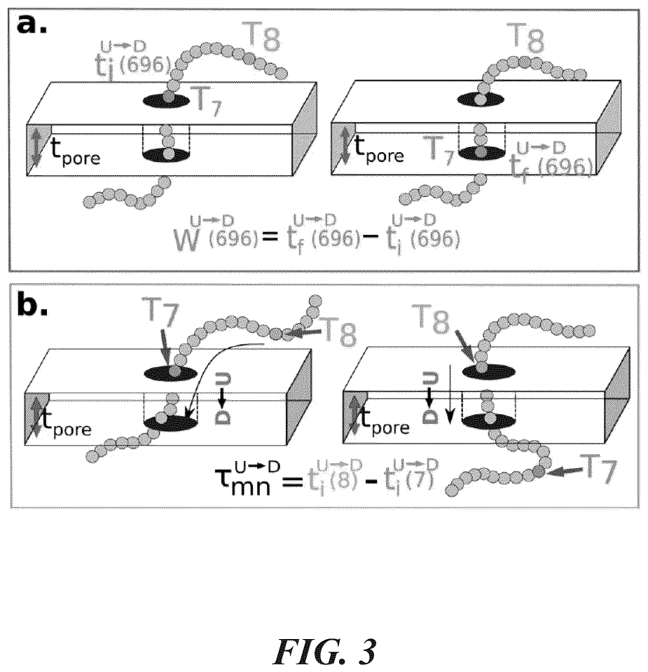

As described above, entry time tᵢ(m) and/or exit time t_{f}(m) of each tag 22 and/or monomer with index m may be recorded as the monomer/tag passes through the nanopore 16 membrane during each scan event, resulting in a calculation of the dwell time W(m). As shown in FIG. 3, the dwell time W(m) for a monomer/tag may be hereby obtained from the difference between the exit and arrival time as:

$${W}^{U \to D} ( m ) = {t}_{f}^{U \to D} ( m ) - {t}_{i}^{U \to D} ( m ) \qquad ( 1 a )$$

$${W}^{D \to U} ( m ) = {t}_{f}^{D \to U} ( m ) - {t}_{i}^{D \to U} ( m ) \qquad ( 1 b )$$

Where tᵢ^{U→D}(m) and t_{f}^{U→D}(m) may represent the arrival and exit times of a monomer with index m through nanopore 16 traveling in a downward, as shown in FIG. 3 (for example, the dwell time calculation for tag 22 T₇ which has a monomer index m=696 is shown in detail in FIG. 3). In addition, the dwell velocities of all tags 22 v_{dwell}(m) for upward and/or downward translocation of the dsDNA segment through nanopore 16 having a defined length along a central axis thereof (i.e., the distance between opposing longitudinal walls 12) of tₚₒᵣₑ may be calculated based on the following dwell time information, in which U→D may represent the downward translation and/or D→U may represent the upward translation:

$${v}_{dwell}^{U \to D} ( m ) = {t}_{pore} / {W}^{U \to D} ( m ) \qquad ( 2 a )$$

$${v}_{dwell}^{D \to U} ( m ) = {t}_{pore} / {W}^{D \to U} ( m ) \qquad ( 2 b )$$

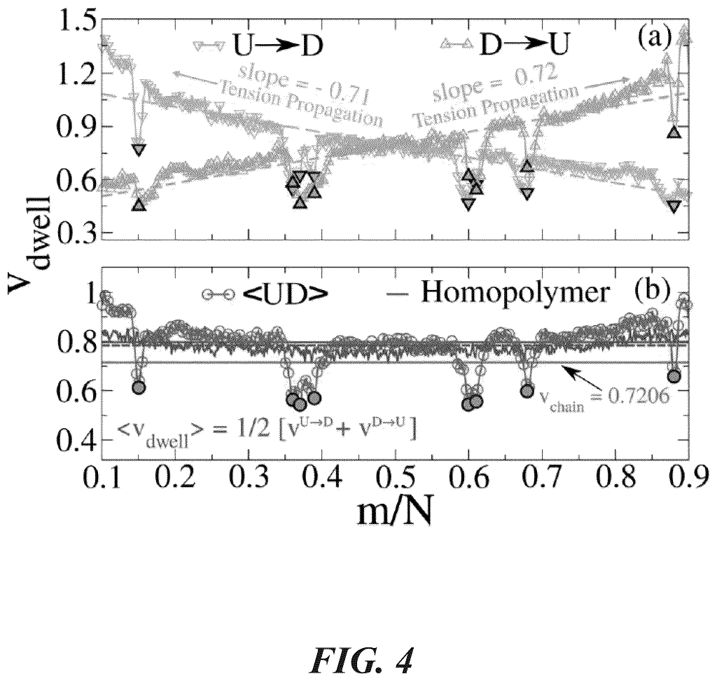

The presence of tags with heavier mass (m_{tag}>m_{bulk}) and/or larger solvent friction (γ_{tag}>γ_{bulk}) may introduce a large variation in the dwell time and/or, hence, a large variation in the dwell velocities of the dsDNA monomers and/or tags, as shown in FIG. 4 (downward triangles for downward dwell velocities, upward triangles for upward dwell velocities, and/or circles for averaged dwell velocities; in addition, filled triangles and/or circles correspond to dwell velocities for tags 22, while empty triangles and circles correspond to monomer velocities). In general, there may be no up-down symmetry for the dwell time/velocity as tags 22 may not be located symmetrically along the chain backbone. Thus, the physical quantities may be averaged over U→D and/or D→U translocation data. The average dwell velocity, calculated as:

$${\overline{v}}_{dwell} ( m ) = \frac{1}{2} [ {v}_{dwell}^{U \to D} ( m ) + {v}_{dwell}^{D \to U} ( m ) ] \qquad ( 2 c )$$

As shown in FIG. 4, which show two different velocity envelopes—the tags residing at the lower envelope.

If the dsDNA were a rigid rod, then the barcode distance (dₘₙ^{U→D}) between tags Tₘ and Tₙ may be calculated by:

$${d}_{mn}^{U \to D} = {v}_{mn}^{U \to D} \times {τ}_{mn}^{U \to D} \qquad ( 3 a )$$

$${v}_{mn}^{U \to D} = \frac{1}{2} [ {v}_{dwell}^{U \to D} ( m ) + {v}_{dwell}^{U \to D} ( n ) ] \qquad ( 3 b )$$

$${τ}_{mn}^{U \to D} = ( {t}_{i}^{U \to D} ( n ) - {t}_{i}^{U \to D} ( m ) ) \qquad ( 3 c )$$

For U→D translocation; the same set of equations may be derived for D→U translocation by interchanging the indices U to D and/or vice versa. Equations 3a-3c may provide the shortest distance between the tags, but not necessarily the contour length, or the actual distance, between the tags. As such, such a calculation may likely be to provide an underestimation of the barcodes.

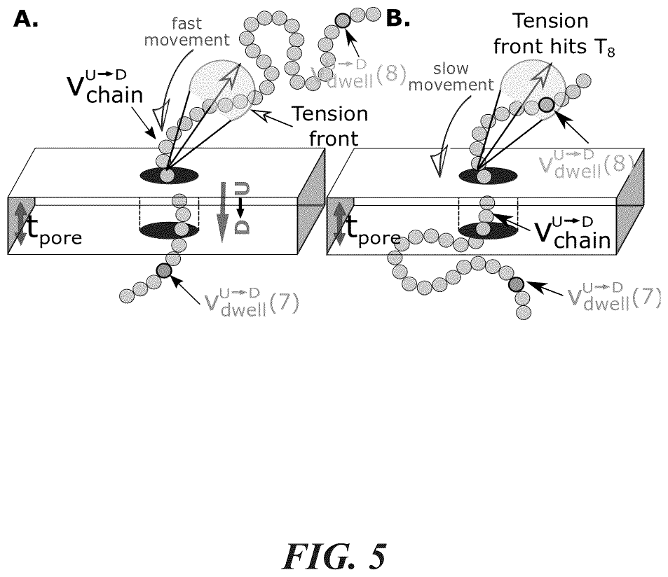

Unlike a rigid rod, tension propagation is important in the semi-flexible dsDNA chain's motion in the presence of an external bias force, as the motion of the dsDNA sub-chain in the cis side decouples into two domains [8, 9]. In an embodiment, as the dsDNA travels through the nanopore 16, after the tag 22 Tₘ translates through the nanopore 16, the preceding monomers may be quickly dragged into the nanopore 16 quickly by the tension front of the dsDNA, similar to an uncoiling effect of a rope pulled from one end. As such, faster motion may occur as the monomer strand translates through the nanopore 16, hitting a maximum at the subsequent tag 22 T_{m±1} with greater inertia and/or viscous drag. In this embodiment, at this tension propagation time, the faster motion of the monomers (e.g., shown in FIG. 5) may begin to taper down to the velocity of the tag 22 T_{m±1}. This process may then continue from one segment to the other. Equations 3a-3c do not account for these contour lengths of faster moving segments in between sequential tags 22, leading to an underestimation of tags 22 and mischaracterization of the DNA barcode.

Accordingly, in an embodiment, a first improved method for accurately determining tag 22 locations, without underestimations, may include measuring a barcode from known end-to-end tag 22 distances. By adding additional tags 22 disposed at the approximate ends of a dsDNA chain or by considering two end tags 22 (T₁ and T₈, with a distance therebetween being defined as d₁₈≃L), an average velocity for the dsDNA chain may be calculated by:

$${v}_{chain}^{U \to D} \approx {v}_{18}^{U \to D} = {d}_{18} / {τ}_{18}^{U \to D} \qquad ( 4 )$$

Where τ₁₈^{U→D} may represent the time delay of arrival for tags 22 T₁ and/or T₈ at the nanopore 16 for U→D scan direction. The barcode distance between tags 22 Tₘ and/or Tₙ may then be calculated by multiplying the time delay with the v₁₈^{U→D} velocity:

$${d}_{mn}^{U \to D} = {v}_{18}^{U \to D} \times {τ}_{mn}^{U \to D} \qquad ( 5 )$$

The method is effective for estimating long-spaced barcodes; however, the method may be prone to overestimate barcode distances if multiple tags 22 are next to each other.

As such, in an embodiment, a second improved method including a two-step process may be employed to correct for overestimations using the average scan time for the entire time, measured experimentally, to estimate the average velocity of the dsDNA chain. The scan length L_{scan} may be the maximum length up to which the dsDNA segment (e.g., including monomers and tags 22) remains captured inside nanopore 16 for scanning events. The scan length may denote the theoretical maximum beyond which the dsDNA will escape from the nanopore 16, L≈L_{scan}. The average scanning velocity from a number of repeated scans, such as 500 independent scans, may be calculated by Equation 6:

$${\overline{v}}_{scan} = \frac{1}{{N}_{scan}} {\sum }_{i} {L}_{scan} / {τ}_{scan} \qquad ( 6 )$$

Where τ_{scan}(i) may represent the scan time for the i^{th} event, N_{scan} may represent the number of scanning events, and/or the average chain velocity may represent v_{chain}≈v̅_{scan}. Using the results from the calculations for normal monomers moving with v̅_{scan}, while tag 22 particles each include respective dwell velocities, the segment velocity between two tags 22 may be estimated by taking the weighted average of the velocities from both tags 22 and normal monomers.

During the first step of the method, the barcode distance between Tₘ and Tₙ may be calculated using only tag velocities v_{dwell}(m) and v_{dwell}(n), using Equations 3a-3c. The estimated distance dₘₙ may then be used to approximately calculate the number of monomers Nₘₙ=dₘₙ^{U→D}/ [symbol] b₁ [symbol] present in a segment joining the two tags Tₘ and Tₙ, with [symbol] b₁ [symbol] being the bond-length. In the second step, the segment velocity may be re-calculated by accounting weighted velocity contribution from both tag 22 and non-tag counterpart as:

$${v}_{weight}^{U \to D} = \frac{1}{{N}_{mn}} [ {v}_{dwell}^{U \to D} ( m ) + {v}_{dwell}^{U \to D} ( n ) + ( {N}_{mn} - 2 ) {\overline{v}}_{scan} ] \qquad ( 7 )$$

The same set of equations for D→U direction may be obtained by interchanging U with D. The barcodes may be finally calculated by multiplying the weighted two-step velocity by the tag time delay as:

$${d}_{mn}^{U \to D} = {v}_{weight}^{U \to D} \times {τ}_{mn}^{U \to D} \qquad ( 8 )$$

The two-step method may accurately capture barcode distances across the range of the dsDNA segment, independent of the proximity of the sequential tags. The underlying concept used in the single nanopore case may be equally applicable to other multi-nanopore systems which use the dwell time and time of flight velocities to measure the barcodes.

### Experimental Results

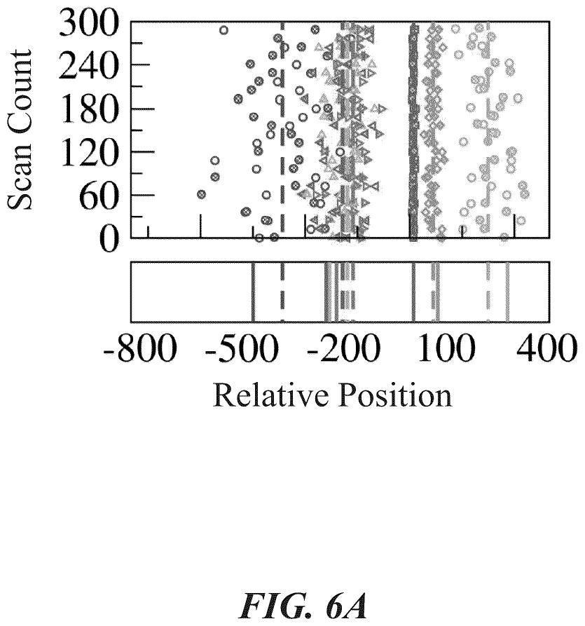

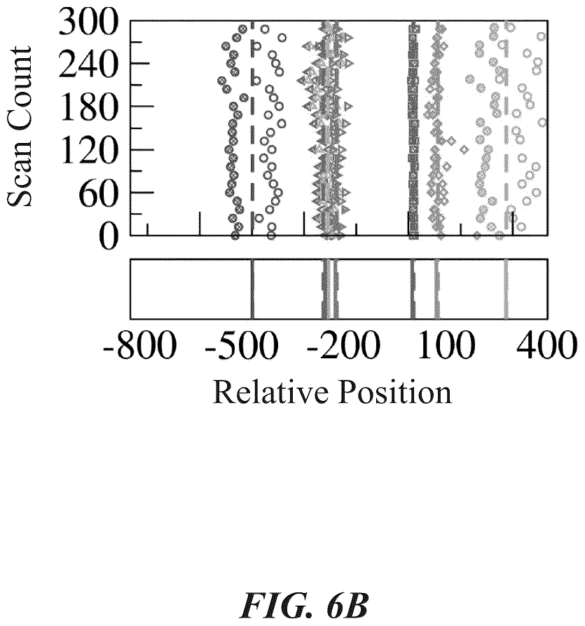

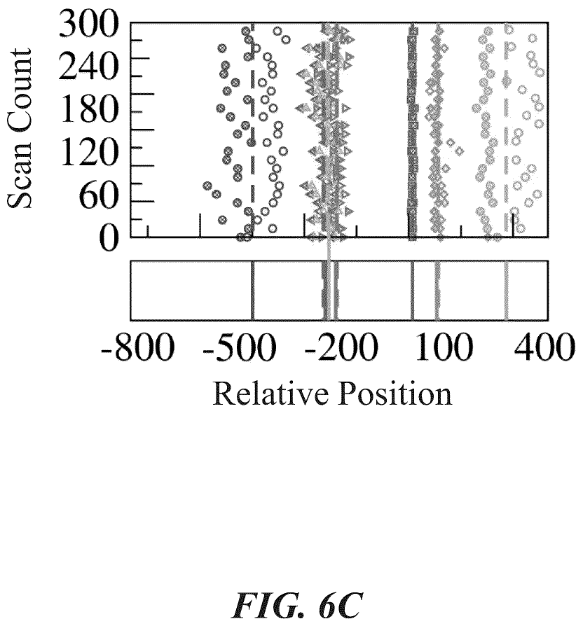

To test the methods described herein, an in silico coarse-grained (CG) model of a dsDNA segment including 1,024 monomers interspersed with 8 barcodes at different distances shown in FIG. 1, approximately mimicking previous studies on longer dsDNA segments (e.g., Zhang et al. including a dsDNA segment with 48,000 base pairs and protein tags of 75 base pairs used as barcodes) [10-12]. The positions of the 8 barcodes (as shown in TABLE 1) were chosen to study whether disparate distances among barcodes affects measurements and accuracies. The tags were introduced by choosing the mass and friction coefficient at tag locations that differ from that of the monomers along the dsDNA chain. The heavier and extended tags introduce a larger viscous drag as compared with the lighter monomers. Moreover, instead of explicitly putting side-chains at the tag locations, the mass and the friction coefficient of the tags were generated to be three times larger than similar measurements of the monomers, providing sufficient information to determine the distance between the tags. FIGS. 6A-6C show simulation results of barcodes generated from the dwell velocity of tags 22 in a single nanopore 16 device using: Equations 3a-3c (shown in FIG. 6A); the single-step method described in detail above (shown in FIG. 6B); and the two-step method described in detail above (shown in FIG. 6C). Furthermore, Table 2 shows the underlying data from the graphical depictions of FIGS. 6A-6C (the abbreviation w.r.t in TABLE 2 denotes “with respect to,” such that the positions of each tag 22 in Table 2 is measured with respect to T₅).

TABLE 1

| Tag # | T₁ | T₂ | T₃ | T₄ | T₅ | T₆ | T₇ | T₈ |
|---|---|---|---|---|---|---|---|---|
| Position | 154 | 369 | 379 | 399 | 614 | 625 | 696 | 901 |
| Separation | 154 | 215 | 10 | 20 | 215 | 11 | 71 | 205 |

TABLE 2
Barcodes measured from different methods

| | Relative | Method of | | |
|---|---|---|---|---|
| | Distance | Equations | One-Step | Two-Step |
| Tag # | w.r.t T5 | 3a-3c | Method | Method |
| T₁ | 460 | 373 ± 122 | 459 ± 59 | 460 ± 43 |
| T₂ | 245 | 197 ± 67 | 250 ± 39 | 250 ± 32 |
| T₃ | 235 | 183 ± 63 | 237 ± 38 | 237 ± 32 |
| T₄ | 215 | 167 ± 54 | 211 ± 35 | 211 ± 30 |
| T₅ | 0 | 0 | 0 | 0 |
| T₆ | 11 | 11 ± 3 | 14 ± 4 | 11 ± 3 |
| T₇ | 82 | 68 ± 23 | 86 ± 23 | 86 ± 21 |
| T₈ | 287 | 230 ± 73 | 287 ± 65 | 287 ± 73 |

### Conclusion

By implementing the barcode determination method described above, utilizing an in-silico Brownian dynamics scheme on a model dsDNA with known locations of the barcodes, a broad distribution of DNA tags may be accurately identified for species classification without overestimation and/or underestimation issues. The method may include the scanning of dsDNA through a cylindrical nanopore multiple times and/or uses the dwell time data of the tags in conjunction with a weighted extrapolation scheme to calculate the average velocities of the chain segment in between two tags. Using one of the tags as a reference, the barcodes may be calculated multiplying time delays between sequential tags by the corresponding segment velocities using Equation 6 and Equation 7.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

### INCORPORATION BY REFERENCE

[1] R. Vernooy, E. Haribabu, MR. Muller, et al, PLOS Biol. 8(7), e1000417 (2010).
[2] N. J. Besansky, D. W. Severson, and M. T. Ferdig, Trends in Parasitology, 19, 545, (2003).
[3] N. Techen, I. Parveen, Z. Pan, and I. A Khan, Current Opinion in Biotechnology, 25, 103 (2014).
[4] X. Xiong, F. Yuan, M. Huang, L. Lu, X. Xiong, and J. Wen, J Food Prot, 82, 1200 (2019).
[5] E. H. -K. Wong, R. H. Hanner, Food Research International, 41, 828 (2008).
[6] P. D. N. Hebert, S. Ratnasingham, and J. R. de Waard, Proc R Soc Biol Sci Ser B, 270, 96 (2003).
[7] S. Pud, S. Chao, M. Belkin, D. Verschureren, T. Huijben, C. van Engelenburg, C. Dekker, and A. Aksimentiev, Nano Lett. 16, 8021 (2016).
[8] T. Sakaue, Phys. Rev. E 76, 021803 (2007).
[9] T. Ikonen, A. Bhattacharya, T. Ala-Nissila and W. Sung, J. Chem. Phys. 137, 085101 (2012).
[10] Y. Zhang, X. Liu, Y. Zhao, J. K. Yu, W. Reisner, and W. B. Dunbar, Small 14, 1801890 (2018).
[11] X. Liu, Y. Zhang, R.Nagel, W. Reisner, W. B. Dunbar, Small 15, 1901704 (2019).
[12] X. Liu, P. Zimny, Y. Zhang, A. Rana, R. Nagel, W. Reisner, and W. B. Dunbar, Small 16, 1905379 (2020).

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. A method of calculating a distance between sequential protein tags within a segment of double-stranded DNA, the method comprising the steps of:
passing the segment of double-stranded DNA through a singular cylindrical nanopore formed within a test chamber, the segment of double-stranded DNA including a first protein tag, a subsequent protein tag, or both;
calculating a weighted velocity of the segment of double-stranded DNA using a dwell velocity for each of a first protein tag and a subsequent protein tag and an estimated number of monomers of a plurality of monomers of the segment of double-stranded DNA; and
calculating the distance between the first protein tag and the subsequent protein tag by multiplying a weighted velocity of the segment of double-stranded DNA by a time delay between the entry time of the first protein tag and the entry time of the subsequent protein tag, the weighted velocity calculated using the dwell velocity.

2. The method of claim 1, further comprising the step of, calculating an average scanning velocity of the segment of double-stranded DNA by dividing a length of the segment of double-stranded DNA by an average scanning time for the double-stranded DNA taken for multiple scans.

3. The method of claim 1, further comprising the step of, calculating an estimated distance between the first protein tag and the subsequent protein tag of the segment of double-stranded DNA by measuring, for the first protein tag and the subsequent protein tag, a dwell time and the dwell velocity based on an entry time into the singular cylindrical nanopore and an exit time from the singular cylindrical nanopore.

4. The method of claim 3, further comprising the step of, using the estimated distance between the first protein tag and the subsequent protein tag, calculating the estimated number of monomers of the plurality of monomers that are disposed between the first protein tag and the subsequent protein tag.

5. The method of claim 4, further comprising the step of, calculating a weighted velocity of the segment of double-stranded DNA using the dwell velocity for each of the first protein tag and the subsequent protein tag and the estimated number of monomers.

6. The method of claim 1, wherein the test chamber includes two opposing longitudinal walls joined together by two opposing lateral walls, such that the singular cylindrical nanopore is formed between the two opposing longitudinal walls, wherein a central axis of the singular cylindrical nanopore is parallel to each of the two opposing lateral walls.

7. The method of claim 1, wherein the singular cylindrical nanopore has an associated diameter of 2σ, where σ is a diameter of each of the plurality of monomers, the first protein tag, and the subsequent protein tag.

8. The method of claim 5, wherein the weighted velocity of the segment of double-stranded DNA is calculated using
$${v}_{weight}^{U \to D} = \frac{1}{{N}_{mn}} [ {v}_{dwell}^{U \to D} ( m ) + {v}_{dwell}^{U \to D} ( n ) + ( {N}_{mn} - 2 ) {\overline{v}}_{scan} ] ,$$
where v_{weight}^{U→D} is the weighted velocity in a downward direction through the singular cylindrical nanopore, Nₘₙ is the estimated number of monomers of the plurality of monomers, v_{dwell}^{U→D}(m) is the dwell velocity of the first protein tag in the downward direction through the singular cylindrical nanopore, v_{dwell}^{U→D}(n) is the dwell velocity of the subsequent protein tag in the downward direction through the singular cylindrical nanopore, and v̅_{scan} is the calculated average scanning velocity of the segment of double-stranded DNA.

9. The method of claim 8, further comprising a step of passing the segment of double-stranded DNA through the singular cylindrical nanopore in an opposing direction.

10. The method of claim 9, further comprising a step of calculating the weighted velocity of the segment of double-stranded DNA in an upward direction through the singular cylindrical nanopore using
$${v}_{weight}^{D \to U} = \frac{1}{{N}_{mn}} [ {v}_{dwell}^{D \to U} ( m ) + {v}_{dwell}^{D \to U} ( n ) + ( {N}_{mn} - 2 ) {\overline{v}}_{scan} ] .$$

11. The method of claim 1, further comprising a step of retaining at least a portion of the segment of double-stranded DNA within the singular cylindrical nanopore throughout each of the multiple scans.

12. The method of claim 1, further comprising a step of passing the segment of double-stranded DNA through the singular cylindrical nanopore in an opposing direction and repeating the steps of calculating the average scanning velocity, calculating the estimated distance between the first protein tag and the subsequent protein tag, calculating the estimated number of monomers of the plurality of monomers, calculating the weighted velocity of the segment of double-stranded DNA, and calculating the distance between the first protein tag and the subsequent protein tag.

13. The method of claim 9, further comprising a step of applying a bias voltage to the test chamber in a reverse direction prior to passing the segment of double-stranded DNA through the singular cylindrical nanopore in the opposing direction.

14. The method of claim 1, further comprising repeating the steps of calculating a distance between sequential protein tags for a plurality of protein tags within the segment of double-stranded DNA.

15. A system for automatically calculating a distance between sequential protein tags within a segment of double-stranded DNA, the system comprising:
a test chamber, the test chamber comprising a computing device having a processor; and
a non-transitory computer-readable medium operably coupled to the processor, the computer-readable medium having computer-readable instructions stored thereon that, when executed by the processor, cause the system to automatically calculate a distance between sequential protein tags within a segment of double-stranded DNA by executing instructions comprising:
passing the segment of double-stranded DNA through a singular cylindrical nanopore formed within the test chamber, the segment of double-stranded DNA including a first protein tag, a subsequent protein tag, or both;
calculating a weighted velocity of the segment of double-stranded DNA using a dwell velocity for each of a first protein tag and a subsequent protein tag and an estimated number of monomers of a plurality of monomers of the segment of double-stranded DNA; and
calculating the distance between the first protein tag and the subsequent protein tag by multiplying a weighted velocity of the segment of double-stranded DNA by a time delay between the entry time of the first protein tag and the entry time of the subsequent protein tag, the weighted velocity calculated using the dwell velocity.

16. The system of claim 15, wherein the test chamber includes a first longitudinal wall disposed at the first side opposite a second longitudinal wall disposed at the second side, with two opposing lateral walls joining the first longitudinal wall to the second longitudinal wall, such that the singular nanopore is formed between the two opposing longitudinal walls, wherein a central axis of the singular nanopore is parallel to each of the two opposing lateral walls.

17. The system of claim 15, wherein the singular nanopore is cylindrical in shape.

18. The system of claim 17, wherein the singular nanopore has an associated diameter of 2σ, where σ is a diameter of each of the plurality of monomers, the first protein tag, and the subsequent protein tag.

19. The system of claim 15, wherein the executable instructions further comprise the steps of:
calculating an average scanning velocity of the segment of double-stranded DNA by dividing a length of the segment of double-stranded DNA by an average scanning time for the double-stranded DNA between the first side of the singular nanopore and the second side of the singular nanopore; and
calculating an estimated distance between a first protein tag and a subsequent protein tag of the segment of double-stranded DNA by measuring, for the first protein tag and the subsequent protein tag, a dwell time and a dwell velocity based on an entry time into the singular nanopore and an exit time from the singular nanopore.

20. The system of claim 19, wherein the weighted velocity of the segment of double-stranded DNA is calculated using
$${v}_{weight}^{U \to D} = \frac{1}{{N}_{mn}} [ {v}_{dwell}^{U \to D} ( m ) + {v}_{dwell}^{U \to D} ( n ) + ( {N}_{mn} - 2 ) {\overline{v}}_{scan} ] ,$$
where v_{weight}^{U→D} weight is the weighted velocity in a downward direction through the singular nanopore, Nₘₙ is the estimated number of monomers of the plurality of monomers, v_{dwell}^{U→D}(m) is the dwell velocity of the first protein tag in the downward direction through the singular nanopore, v_{dwell}^{U→D}(n) is the dwell velocity of the subsequent protein tag in the downward direction through the singular nanopore, and v̅_{scan} is the calculated average scanning velocity of the segment of double-stranded DNA.
